(19) United States
(12) Patent Application Publication
(10) Pub. No.: US 2025/0170379 A1
(43) Pub. Date: May 29, 2025

(54) Melting Microneedle Patches and Methods of Manufacturing Thereof

(71) Applicant: Georgia Tech Research Corporation, Atlanta, GA (US)

(72) Inventors: Yasmine Abdou GOMAA, Alpharetta, GA (US); Thy C. NGUYEN, Jonesboro, GA (US); Mark R. PRAUSNITZ, Atlanta, GA (US)

(21) Appl. No.: 18/841,936

(22) PCT Filed: Feb. 28, 2023
(86) PCT No.: PCT/US2023/014179

§ 371 Date: Aug. 27, 2024

Related U.S. Application Data
(60) Provisional application No. 63/314,835, filed on Feb. 28, 2022.

(51) Int. Cl. A61M 37/00; A61K 9/00; A61K 31/573; A61K 47/14

(52) U.S. Cl. CPC A61M 37/0015; A61K 9/0021; A61K 31/573; A61K 47/14; A61M 2037/0023; A61M 2037/0053; A61M 2037/0061

(57) ABSTRACT

Microneedles for drug delivery that include a wax or other meltable material and methods of making and using such microneedles are disclosed. A microneedle patch includes a backing layer; and an array of microneedles extending from the backing layer, wherein the microneedles each include a distal tip portion that includes a drug, and the microneedles are configured to be inserted into mammalian tissue and, at least a portion of each of the microneedles is configured to melt in the mammalian tissue. In some other cases, melt materials are used during manufacturing of the microneedles but do not melt in the skin.

### CROSS-REFERENCE TO RELATED APPLICATIONS

This application claims priority to U.S. Provisional Application No. 63/314,835, filed on Feb. 28, 2022, which is incorporated herein by reference.

### BACKGROUND

Microneedle patches have been developed as a minimally invasive means to deliver drugs through the skin using several approaches. Among general approaches of skin delivery using microneedle patches, dissolving microneedle patches are of interest. Dissolving microneedle patches are made of dissolving excipients that tend to dissolve in the interstitial fluid at the insertion site of the skin, thereby releasing the drug payload. However, conventional microneedle-based technologies may have shortcomings that make microneedle patches unsuitable for use in certain circumstances.

For example, poor aqueous solubility of some active pharmaceutical ingredients can be an obstacle to expanding the uses of conventional microneedle patches. A conventional approach for processing drugs with poor aqueous solubility is to use organic solvents in a microneedle casting process, which significantly complicates the manufacturing process with added lengthy evaporation steps that ultimately may destabilize the loaded bioactive materials.

Moreover, the intrinsic instability of biomolecules is also a cause for concern and a predominant challenge for biopharmaceutical commercialization. Because therapeutic proteins have greater conformational mobility when in aqueous solution, liquid formulations of proteins are more susceptible to degradation. Consequentially, water segregation and removal, and embedding proteins in a rigid matrix are good approaches for improved storage stability. While dissolving microneedle patches have demonstrated increased stability of incorporated biomolecules, such as proteins and vaccines, residual moisture content is unavoidable with current drying techniques for existing microneedle patches.

Furthermore, some vaccines require either at least one booster dose to induce lasting immunity. Extended delivery of an inactivated antigen could present antigen over a prolonged period, thereby generating a stronger immune response to the same dose. A common approach for controlled release is to encapsulate the active ingredient in bioerodible polymers, but this approach may not translate well to protein antigens. This is primarily due to the harsh stresses experienced during fabrication and sterilization of, and release from, the biodegradable polymer matrix, which are known to damage sensitive proteins.

Accordingly, there is a need to provide microneedle compositions and patches that overcome or mitigate the foregoing shortcomings of conventional microneedle patches, while also offering additional benefits as compared to existing microneedle-based technologies.

### BRIEF SUMMARY

In one aspect, a microneedle patch is provided that includes a backing layer; and an array of microneedles extending from the backing layer, the microneedles each comprising a distal tip portion which comprises a drug, wherein the microneedles are configured to be inserted into mammalian tissue and at least a portion of each of the microneedles is configured to melt in the mammalian tissue.

In another aspect, a method of making a microneedle is provided, which includes preparing a first composition comprising a drug dispersed in a first excipient material heated to a temperature greater than the melting point of the first excipient material; casting the first composition at a temperature less than 100° C. in a mold having a cavity defining one or more microneedles; and then cooling the first composition in the mold to a temperature that is less than the melting point of the first excipient material to form one or more microneedles, or at least a distal tip portion of one or more microneedles, in the mold.

In still another aspect, a microneedle patch is provided that includes a backing layer; and an array of microneedles extending from the backing layer, the microneedles each comprising a portion which comprises a drug, wherein the microneedles are configured to be inserted into mammalian tissue, and wherein the microneedles are made of one or more materials that melt at a temperature between 60° C. and 100° C.

In yet another aspect, a method of delivering a drug to a patient is provided that includes inserting a microneedle, which comprises a drug, into the patient's skin; and then melting the microneedle, or a distal tip portion thereof, into the patient's skin.

In a further aspect, a method is provided for segregating a drug in a distal tip portion of a microneedle in a microneedle patch from a backing layer of the microneedle patch. The method includes interposing a hydrophobic material in a proximal portion of the microneedle between the backing layer and the distal tip portion. The distal tip portion and/or the proximal portion may be configured to dissolve or melt following insertion into skin.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may or may not be present in various embodiments. Elements and/or components are not necessarily drawn to scale.

FIG. 1 is a perspective view of an array of meltable microneedles, according to one or more embodiments of the present disclosure.

FIG. 2A is a microphotograph showing a perspective view of an array of meltable microneedles, according to one or more embodiments of the present disclosure.

FIG. 2B is a microphotograph showing a close-up of an array of two-cast meltable microneedles, according to one or more embodiments of the present disclosure.

FIG. 3 depicts a process for manufacturing an array of meltable microneedles, according to one or more embodiments of the present disclosure.

FIGS. 4A-4N are schematic, cross-sectional, views of microneedles, illustrating several different design approaches for incorporating a meltable material into the structure of a microneedle, according to one or more embodiments of the present disclosure.

FIG. 5 depicts a mechanism by which melting microneedles deliver a drug into tissue, according to one or more embodiments of the present disclosure.

FIGS. 6A-6D are schematic cross-sectional, views of different approaches for including a meltable portion in a separable microneedle and illustrating that separation following insertion in to tissue, according to one or more embodiments of the present disclosure.

### DETAILED DESCRIPTION

Microneedles, microneedle arrays, and microneedle patches have been developed wherein the microneedle is (1) configured, e.g., has the geometry and mechanical properties, to be inserted into skin (or other mammalian tissue), and (2) configured, e.g., has a suitable composition, to enable all or a portion of the microneedle to melt in the skin (or other mammalian tissue). Such microneedle compositions advantageously may mitigate or eliminate some difficulties associated with conventional manufacturing of microneedle arrays, including maintaining stability of a drug incorporated into the microneedle, and beneficially provide alternative techniques for (1) controlling release of drug from a microneedle-based drug delivery system, (2) facilitating separation of microneedles from a microneedle patch as part of an administration method, and/or (3) eliminating “sharps” following application of a microneedle-based drug delivery system to a patient.

In addition, the methods of making microneedle arrays described herein advantageously, in some embodiments, may enable a drug to be segregated and protected from undesired exposure to water or other environmental components by the inclusion of suitable hydrophobic materials in the construction of a microneedle patch. For example, a drug may be disposed within in a distal tip portion of a microneedle, e.g., in a water-soluble first excipient, in a microneedle patch, and the hydrophobic material is interposed in proximal portion of the microneedles between the backing layer of the patch and the distal tip portion of the microneedles.

As used herein, the terms “melt” and “melting” in reference to a microneedle patch refer to a phase change from solid to liquid of at least part of the composition, e.g., one excipient of a multi-excipient formulation, forming the microneedle patch; it does not mean that every excipient component of the composition necessarily undergoes this phase change.

For example, there may be a dispersed solid component that remains in solid form even though the microneedle has lost its structural integrity upon other components of the composition changing into a liquid. In another example, the drug remains solid and the microneedle matrix material is melted during manufacturing i.e., before/while the microneedle is being formed.

### Microneedle Patches

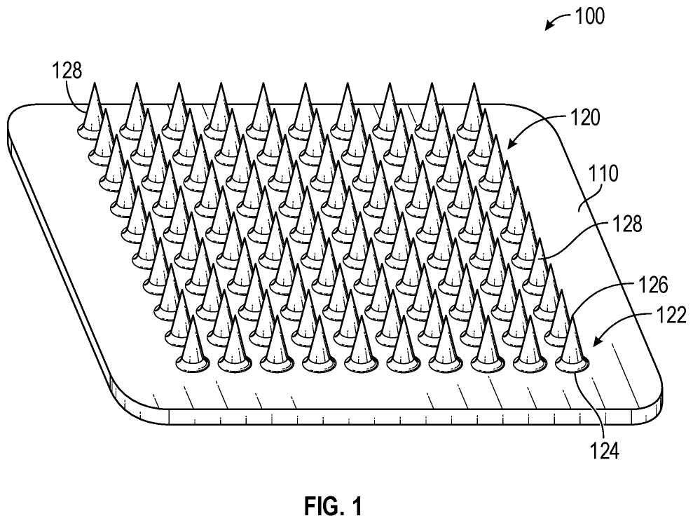

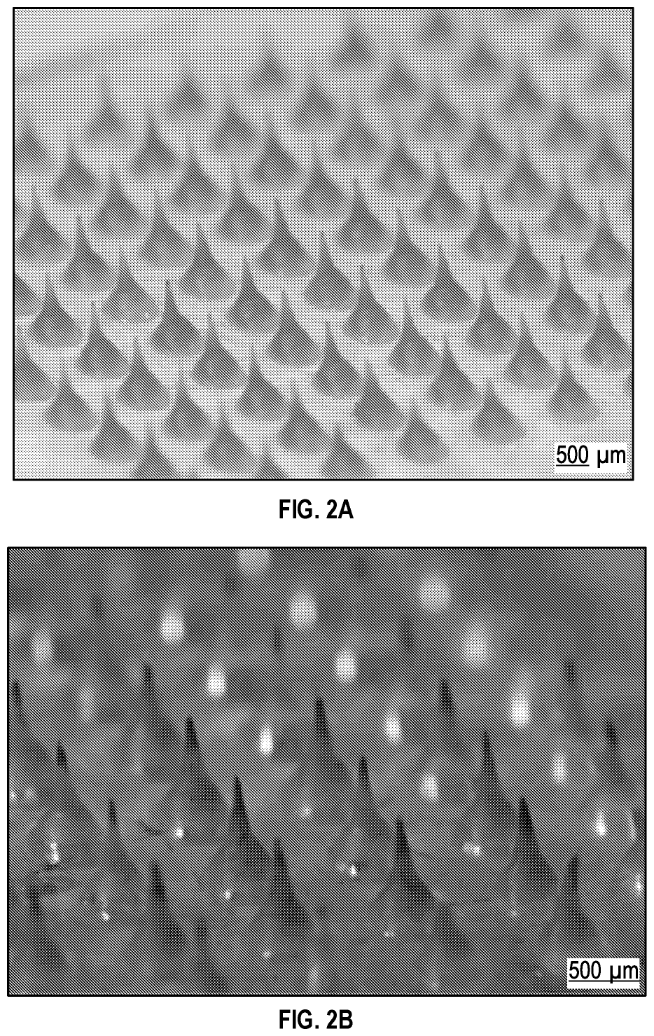

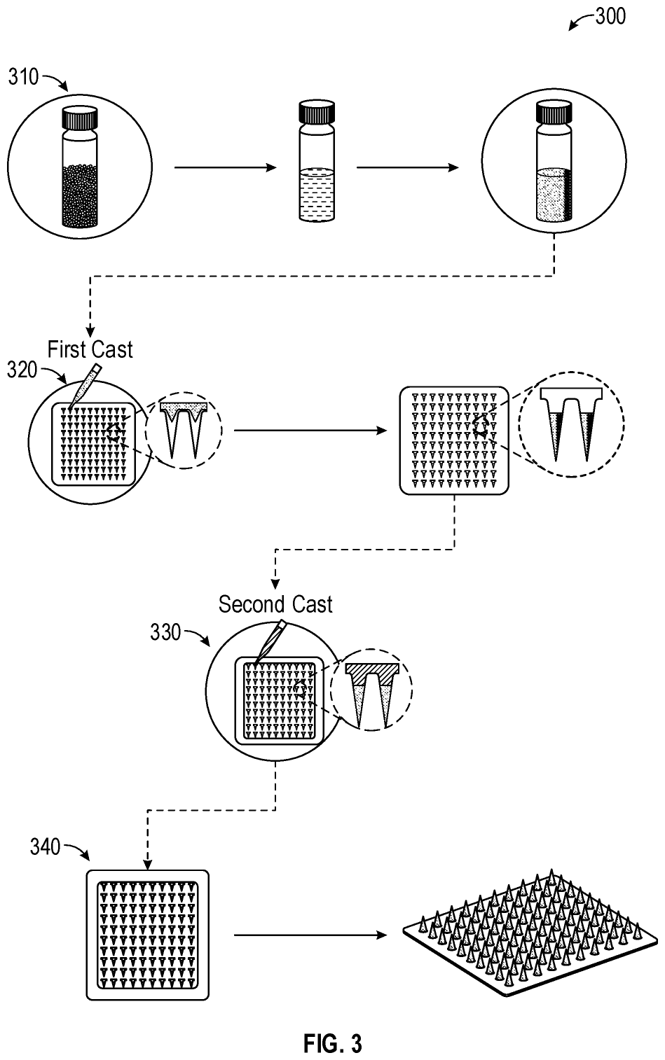

One example of microneedle patch with an array of microneedles is depicted in FIG. 1. The microneedle patch 100 includes a base substrate 110 from which microneedles 120, in a 10×10 array, extend. The phrase “base substrate” and the term “substrate” or “backing” are used interchangeably herein. Each microneedle 120 has a proximal end 122 attached to the base substrate 110 directly, or indirectly, via one or more proximal portions 124, and a distal tip end 126 which is sharp and effective to penetrate biological tissue. The microneedle 120 has tapered sidewalls 128 between the proximal end 122 and distal end 126 of the microneedle 120. Other geometries are possible. As detailed below, at least a part of the microneedles 120, the proximal portion 124, and/or the substrate 110 includes a meltable material. The substrate 110 may remain intact, or some or all of it may melt with all or a portion of the microneedles 120. Exemplary photographs of microneedle arrays comprising a meltable portion as disclosed herein are shown in FIGS. 2A-2B.

The microneedles of the microneedle patch are designed to insert into skin or another biological tissue and then all or part of the microneedle melt in response to heating by (i) heat transfer from the mammalian tissue, and/or (ii) application of heat from an external heat source applied to the inserted microneedles. Essentially any suitable heat sources could be used, for example, applied to the back of the microneedle patch. Examples include heating pads and hand warmers as known in the art. In some embodiments, the heat source may be incorporated within the backing of the microneedle patch, such that the heating may be triggered by pressing on the back of the patch during insertion into the skin to begin the process of warming and melting the emails.

In some embodiments, the length of the microneedle may be between about 50 μm and 2 mm, from about 100 μm to about 2000 μm, from about 100 μm to about 1500 μm, from about 200 μm to 1000 μm, or ideally between about 500 μm and 1000 μm. In some embodiments, the array of microneedles includes from 10 to 1000 microneedles, from 10 to 500 microneedles, from 10 to 250 microneedles, from 50 to 250 microneedles, or 100 microneedles (e.g., a 10-by-10 array of microneedles).

In some embodiments, the microneedle includes a wax, such as a glycerol ester of fatty acid such as found in Witespol, or a fatty acid such as stearic acid, lauric acid, etc. In some preferred embodiments, the wax is designed to melt following insertion of the microneedle into human skin and reaching or exceeding the skin temperature of a human, to cause the microneedle to lose its structure and at least in some cases separate from the base. For example, the wax may include mono, di, and triglycerides of C8-C20 fatty acids such capric acid (C10), pelargonic acid (C9), undecylic acid (C11), lauric acid (C12), tridecylic acid (C13), myristic acid (C14), pentadecylic acid (C15), palmitic acid (C16), palmitoleic acid (C16:1), margaric acid (C17), stearic acid (C18), oleic acid (C18:1), linoleic acid (C18:2), nonadecylic acid (C19), and/or arachidic acid (C20), and/or vegetable oils such as coconut oil, palm kernel oil, palm oil, and/or palm stearin which are composed of mixtures of fatty acids.

In some preferred embodiments, the microneedles include a hydrophobic matrix material in which a drug is dissolved or dispersed, wherein the matrix material is designed to melt following insertion of the microneedle into human skin. The matrix material upon reaching or exceeding the skin temperature of a human, which may be about 30° C. to 38° C., then becomes a liquid to cause the microneedle to lose its structure and at least in some cases separate from the base. The drug is then released from the matrix material into the tissue at the site of insertion. For instance, the drug may partition into the surrounding interstitial fluid while and/or after the hydrophobic matrix is melted by the skin. The degree to which the drug partitions into the interstitial fluid may depend on the intrinsic aqueous solubility of the drug. For example, a highly water-soluble drug may partition into the interstitial fluid at a faster rate than a less water-soluble drug. This system is particularly advantageous for delivering poorly water-solubilized drugs, or water-insoluble drugs, which are typically difficult to administer using existing microneedle technology.

In some embodiments, the hydrophobic matrix comprises a so-called fatty suppository base. Fatty suppository bases are known in the art to be safe for rectal insertion for drug administration to the human body, and may offer additional localized benefits, such as skin moisturization. A wide selection of suppository bases can be used individually or in combination with other bases to obtain the optimal drug-release profile for different applications as described herein. Example of suitable fatty suppository bases include carnuba wax, beeswax, Kolliwax CA (BASF); Novata BCF PH, BC PH, or B PH (BASF); Wecobee M or FS (Stephan); Witepsol H 15 (Fagron); Witespol W32, W45, S55, E75, E76, E85, or S58 (IOI Oleo GmbH); HydroKote 112 or M (Abitec); or Suppocire NA15, AML, D, BM, AS2, or A (Gattefose). In a preferred embodiment, the fatty suppository base is Witepsol S55 (IOI Oleo GmbH). The terms and phrases “drug” and “substance of interest” (referred to as a “SOI”) are used interchangeably herein, unless otherwise expressly indicated. The drug may be fully or partially soluble or insoluble in the matrix material forming the microneedle. In some embodiments, the drug is solubilized with the fatty suppository base by heating the base above its melting point, and adding the drug to the melted material. In some other embodiments, an insoluble drug is dispersed into the melted fatty suppository base without the use of organic solvents, or other additives to improve solubility.

In some embodiments, the SOI is a cosmetic agents, cosmeceuticals, diagnostic agents, markers and other materials that are desirable to introduce into a biological tissue. In some embodiments, the drug is a prophylactic or therapeutic substance, which may be referred to herein as an active pharmaceutical ingredient (API). The API may be selected from suitable proteins, peptides and fragments thereof, which can be naturally occurring, synthesized or recombinantly produced.

In some embodiments, the drug is a hormone or steroid. The hormone may be a contraceptive hormone known in the art. In one example, the drug comprises levonorgestrel.

In some embodiments, the drug comprises a vaccine. Examples of vaccines include vaccines for infectious diseases, therapeutic vaccines for cancers, neurological disorders, allergies, and smoking cessation or other addictions.

In some other embodiments, the drug comprises a therapeutic agent. The therapeutic agent may be selected from small molecules and larger biotechnology produced or purified molecules (e.g., peptides, proteins, DNA, RNA, aptamers).

In yet another embodiment, the SOI is a vitamin, herb, or dietary supplement known in the art.

In some embodiments, the microneedles within a given array of microneedles all contain the same drug and excipient. In other embodiments, the microneedles within a given array of microneedles may contain different drugs and/or excipients. For example, the drugs and/or the excipients may be different in each microneedle, in different rows of microneedles, or sections/regions of the microneedle array. Possible reasons for designing the microneedles with such segregation are: i) the different drugs are incompatible with one another, ii) the different drugs require different stabilizing excipients, and iii) different release profiles (e.g., combination of rapid bolus followed by a sustained release) are desired of a single drug or of different drugs.

The drug desirably is provided in a stable formulation or composition (i.e., one in which the biologically active material therein essentially retains its physical stability and/or chemical stability and/or biological activity upon storage). Stability can be measured at a selected temperature for a selected period. Trend analysis can be used to estimate an expected shelf life before a material has actually been in storage for that time period. Because the melting microneedle patches disclosed herein exclude water from at least part of the casting process, stability may be improved to allow for extended storage time outside the cold chain. Moreover, formulating the drug into melting microneedles may effectively extend the release of the drug without the need for harsh encapsulation conditions otherwise necessary to delay release of drug in dissolving microneedles, for example.

The microneedle patch, may further include other structural elements (not shown in FIG. 1) that enhance storage and usability of the microneedles. For example, the patch may include a housing and/or other layers, such as a handle layer, on the side of the base substrate opposing the microneedles. Examples of such other additional structural components are described in U.S. Pat. No. 10,265,511 to McAllister et al., which is incorporated herein by reference.

### Methods of Making Microneedle Arrays

The arrays of microneedles described herein may be made by any suitable process. In a preferred embodiment, the arrays of microneedles are made using a molding process, which advantageously is highly scalable. The filling and molding steps described herein may be referred to herein as “casting”. In some embodiments, the casting methods, molds, and other equipment may be adapted from those known in the art, such as described in U.S. Pat. No. 10,828,478 to McAllister et al., which is incorporated herein by reference. The methods for making the microneedles preferably are performed under a minimum ISO 7 (class 10,000) process or an ISO 5 (class 100) process.

The process may include preparing a composition with a drug dispersed, or dissolved, in an excipient, such as a fatty suppository base; heating the composition to a temperature above the melting point of the excipient; filling a mold having a cavity defining one or more microneedles; and cooling the composition in the mold to below the melting point of the excipient to form one or more microneedles. Centrifugation and/or vacuum may be used to aid this process, particularly in that the centrifugation and/or vacuum may help maintain microneedle shape as defined by the mold as the drug-excipient composition solidifies. This process based on melting and solidification advantageously eliminates the need for extensive drying steps, which may significantly reduce the duration of the manufacturing process.

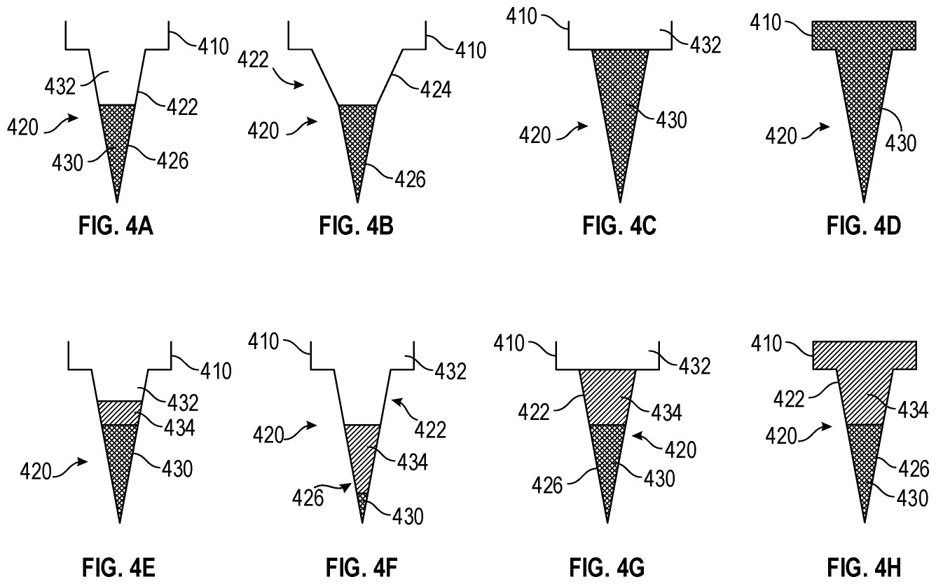

In some embodiments, a two-cast or three-cast process is used. In these embodiments, the first cast of the drug and excipient forms only a distal tip portion of the microneedles. The subsequent cast may involve filling the remaining space of the mold on top of the distal tip portion of the microneedles to form a proximal portion of the microneedles. In some embodiments, a second cast is applied to the microneedle mold prior to removing the microneedles from the mold. In some embodiments, the second cast is of a different composition from the first cast. For example, the second cast may be aqueous-based, so that it could dissolve in vivo concurrently with melting of the first cast composition. For example, aqueous-based casts may be made of a mixture of a polymer, such as polyvinyl alcohol (PVA) or polyvinylpyrrolidone (PVP), and a sugar, such as sucrose. In some preferred embodiments, the second cast is equal parts 18% PVP (360/55) and sucrose. In a preferred embodiment, as shown in FIG. 3, the manufacture (300) of solid, melting microneedles includes at least four steps: (1) forming 310 the first cast composition, (2) the first cast filling 320, (3) the second cast filling 330, and (4) cooling/drying 340. Forming the first cast composition includes the steps of (i) melting an excipient to a temperature above its melting point, and (ii) dissolving or dispersing a drug in the melted excipient to form the first cast composition. While the drug is mixed with the excipient, the temperature remains above the melting point of the excipient to avoid premature congealing or hardening of the composition. In some embodiments, the melting point of the excipient is between 30° C. and 40° C., such that the excipient may be heated to at least 40° C., or preferably 50° C., but still may melt upon insertion to the skin and/or application of an external heat source. In other embodiments, the melting point of the excipient is much higher, such as between 50° C. and 100° C., where the excipient melts during the manufacturing process but the microneedles do not melt when inserted into the skin.

After the first cast composition is formed, it is transferred into a negative mold of the one or more microneedles, typically an array of between 10 and 1000 microneedles. This may be referred to as the “first cast.” The mold may then be centrifuged and/or vacuumed to facilitate displacement of any air bubbles and movement of the composition into the microneedle tips of the mold. The mold may centrifuged at a temperature higher than the melting point of the excipient. In some embodiments, the temperature is less than 50° C., preferably between 40° C. and 25° C. Temperature may range from about 60° C. to about 80° C. for casting high melting point waxes (i.e., waxes that do not melt in the skin). During the first cast of a multi-cast process, the composition may at least partially congeal or harden, but it is not required that the first cast be fully solidified at this point.

The mold is then filled with a second composition that is suitable for forming the backing substrate and, optionally, a proximal end portion of the microneedles. In some embodiments, this second cast material may be or may include one or more water-soluble materials. In some other embodiments, this second cast material may be or may include a meltable material. In some embodiments, this second cast material is drug-free, i.e., contains only one or more excipients. In some embodiments, this second cast material for the proximal part of the microneedle may include a drug that is the same as or different from the drug in the first cast composition. After being filled, the mold may be centrifuged and/or vacuumed to facilitate and expedite the casting process.

The filled molds are permitted to solidify and/or dry as needed. The filled molds optionally may be dried within a desiccant chamber to facilitate removal of excess water or moisture from the patches. After the casts are substantially solidified and/or dry, the melting microneedle patch may be removed from the mold.

In some embodiments, the microneedle patches are manufactured using a three-cast process, where the first cast is a drug-matrix composition forming the distal tip portion of the microneedles, the second cast is an excipient forming a proximal end portion of the microneedles and/or a connector/funnel portion between the microneedles and the base substrate, and the third cast is a composition suitable for forming the base substrate. In these embodiments, the second cast excipients may melt upon insertion, and may serve as a separating layer between the distal tip portion (which may or may not also melt in the skin, or may or may not dissolve in the skin) and the backing substrate (which may or may not also melt in or on the skin, or may or may not dissolve in or on the skin). In some embodiments, it may be preferable to prevent the drug matrix composition and base composition from contacting one another, or mixing. The middle second cast can serve to prevent this interaction. For example, molten wax can be used as a middle second cast in a three-cast microneedle patch manufacturing process. The wax may serve as a hydrophobic barrier between the drug in the microneedles and the backing, preventing the drug from being wetted by water present in the backing during manufacturing and/or afterwards, which could affect drug stability.

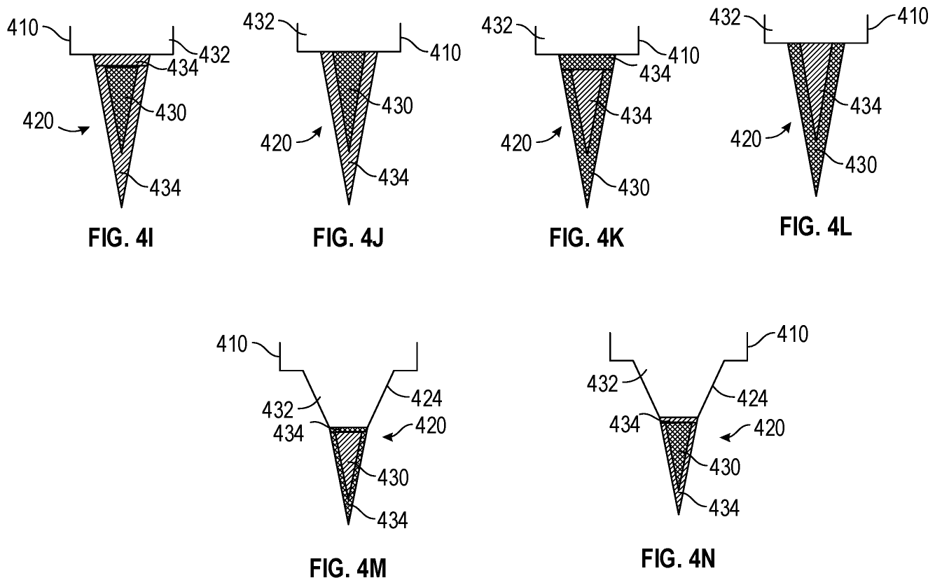

FIGS. 4A-4N illustrate embodiments of different casting arrangements. In FIGS. 4A-4B, the melting microneedle patches are formed from only two casts. In these embodiments, the first cast is formed of a meltable drug-excipient composition 430, while the second cast is formed of a different water-soluble material 432. As shown in FIG. 4A, the first cast formed of the drug-excipient composition 430 may form the distal tip portion 426 of the microneedle 420, and the second cast of the water-soluble base 432 may form the proximal end portion 422 of the microneedle 420 and the substrate 410 from which the microneedle 420 extends. As shown in FIG. 4B, the microneedle 420 may include a base portion 424 at the proximal end 422 of the microneedle 420. This proximal base 424 may be formed of the second cast 432, along with the substrate 410 to which the proximal portion is attached. In FIG. 4A, the microneedle has a continuous taper, and in FIG. 4B the microneedle has a discontinuous taper that forms a funnel. The materials and fabrication process may be the same for both geometries.

In FIGS. 4C-4D, the microneedles 420 are formed out of a single cast of a drug-excipient composition 430. In some embodiments, as shown in FIG. 4C, the microneedle 420 is cast out of the drug-excipient composition 430, and extends from a substrate 410 formed of a cast of a water-soluble base 432. In FIG. 4D, the microneedle 420 and the substrate 410 from which it extends may be formed of the drug-excipient composition 430.

In FIGS. 4E-4H, the melting microneedles 420 are formed out of two or more casts. In these embodiments, the first cast is a drug-excipient composition 430 and the second cast is an excipient 434. The first drug-excipient composition cast 430 forms the distal tip portion 426 of the microneedle 420, while the second excipient cast 434 forms at least a portion of microneedle 420 between the distal tip portion 426 and the proximal end portion 422 of the microneedle. In FIGS. 4E-4F, the microneedle patches include a third cast formed of a water-soluble base 432, where the third cast of the base forms the proximal end portion 422 of the microneedle 420 and the substrate 410 from which the microneedle 420 extends. In other embodiments, as shown in FIG. 4G, the second excipient cast 434 also forms the proximal end portion 422 of the microneedle 420. In further embodiments, as shown in FIG. 4H, the second excipient cast 434 forms the proximal end portion 422 of the microneedle 420 and the substrate 410 from which the microneedle 420 extends.

In FIG. 4I-4L, the melting microneedles are also formed out of two or more casts. In these embodiments, at least two casts form the microneedle 420 such that one or more casts form a coating or shell around a cast of the drug-excipient composition 430. In FIGS. 4I-4J, a first cast of an excipient forms a coating around a second cast of a drug-excipient composition 430. In FIG. 4I, a third excipient 434 cast forms caps the excipient 434 coating and drug-excipient 430 core, such that the excipient 434 casts together form a shell around the drug-excipient composition 430. The substrate 410 from which the microneedle 420 extends is then formed of a water-soluble base 432 as a fourth cast. As with FIG. 4I, FIG. 4J depicts a first excipient 434 cast that forms a coating around a second cast of a drug-excipient composition. However, the substrate 410 formed of a third cast of a water-soluble base 432 is formed directly on top of the excipient 434 coating and drug-excipient 430 core (i.e., the coating and core are not capped with another excipient 434 cast.

In FIGS. 4K-4L, the first cast of an excipient 434 forms a coating around the second cast of a drug-excipient composition 430. In FIG. 4K, a third excipient 434 cast forms a cap in the proximal end portion 422 of the microneedle, such that first and third excipient 434 cases together form a shell around the drug-excipient 430 core of the microneedle 420. The substrate 410 from which the microneedle 420 extends is formed of a water-soluble base 432 fourth cast. In FIG. 4L, the substrate 410 from which the microneedle 420 extends, formed of a water-soluble base 432, is cast directly on top of the excipient 434 coating and drug-excipient 430 core. In FIGS. 4I-4J, the SOI is in the core and not the shell/coating, whereas in FIGS. 4K-4L, the SOI is in the shell/coating but not in the core.

In FIG. 4M-4N, the distal tip portion 426 of the microneedle is formed of three casts, where the first cast forms a coating around the second cast, which is capped using a third cast. In FIG. 4M, a first and third case of the drug-excipient composition 430 forms a coating around a second excipient 434 cast. In FIG. 4N, the first and third excipient 434 casts 434 form a shell around the second SOI composition cast 430. In each of these embodiments, a fourth cast of a water-soluble base 432 is used to form the proximal portion 424 of the microneedles and the substrate 410 from which the microneedles extend.

### Methods of Use

The microneedle arrays described herein may be used to administer a variety of substances into a tissue site in a human or other mammal, typically by applying a microneedle patch that includes an array of the meltable microneedles to a skin surface in a manner to cause the microneedles to penetrate the stratum corneum and enter the viable epidermis and, possibly, the dermis.

As used herein, the phrase “penetrate a tissue surface,” includes penetrating a biological tissue surface with at least the distal tip ends of the microneedles. Upon separation of a microneedle from a substrate, a proximal end of a microneedle may be above a tissue surface, substantially level with a tissue surface, or below a tissue surface.

The phrase “biological tissue,” as used herein, generally includes any human or mammalian tissue. The biological tissue may be the skin or a mucosal tissue of a human or other mammal in need of treatment or prophylaxis. It is envisioned that the present devices and methods may also be adapted to other biological tissues and other animals.

The microneedle patches may be self-administered or administered by another individual (e.g., a parent, guardian, healthcare worker).

The methods described herein further include a simple and effective method of administering a SOI to a patient with a microneedle patch. The method may include identifying an application site and, preferably, sanitizing the area prior to application of the microneedle patch (e.g., using an alcohol wipe). The microneedle patch then is applied to the patient's skin/tissue and manually pressed into the patient's skin/tissue (e.g., using the thumb or finger) or using a device to facilitate patch application so that the microneedles penetrate the tissue surface.

After administration, the substrate (and remaining microneedle patch structure) may be removed from the patient's skin/tissue, at least in embodiments where the substrate remains intact.

In some embodiments, the microneedle patches described herein are used to deliver one or more SOIs into the skin. In one embodiment, the microneedle patches are used to deliver the SOI into skin by inserting the microneedles across the stratum corneum (outer 10 to 20 microns of skin that is the barrier to transdermal transport) and into the viable epidermis and possibly the dermis. The small size of the microneedles enables them to cause little to no pain and target the intracutaneous space. The intracutaneous space is highly vascularized in the dermis and rich in immune cells in the dermis and epidermis, and provides an attractive path to administer both vaccines and therapeutics. The microneedles may melt once in the intracutaneous space to release the SOI into the interstitial fluid and skin.

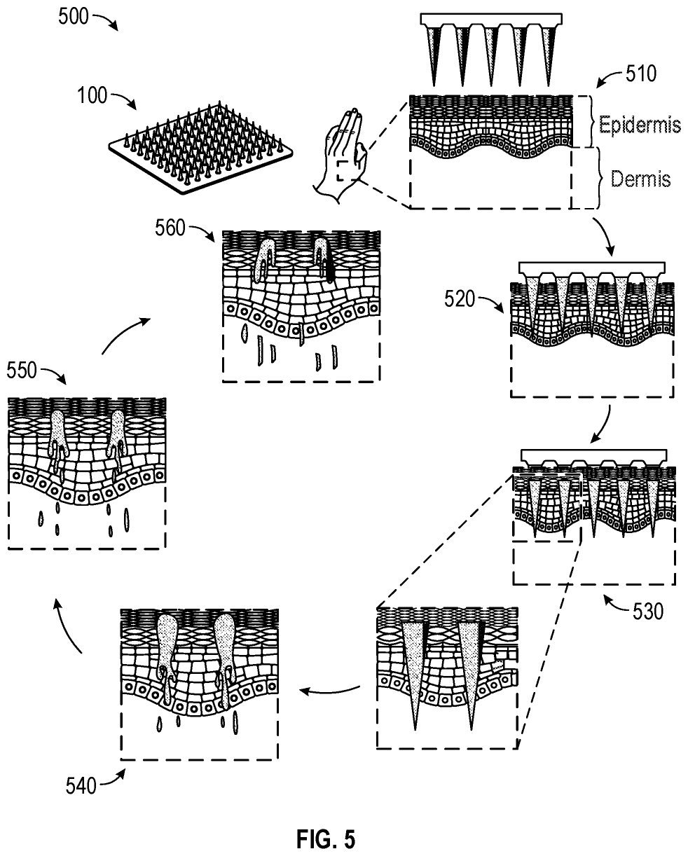

FIG. 5 illustrates one method (500) for administering a SOI to a patient. The method includes providing a microneedle patch 100 as described herein; aligning the microneedle patch with a target site at the skin surface (step 510); and inserting the microneedles of the microneedle patch into the skin (step 520) to force the microneedles through at least the stratum corneum and into the viable epidermis and possibly the dermis layers of the skin. Subsequently, the microneedles are shown to separate from the base (step 530) and the microneedles begin to melt (step 540). Separation may occur, for example, quickly after insertion when the substrate and/or proximal ends of the microneedles are formed of water-soluble material, enabling removal of base within a few seconds or minutes after microneedle insertion (and before completion of melting and drug release). Melting and dissipation of the microneedle composition continue (steps 550 and 560).

Although the microneedles are shown to separate from the base before microneedles melt, this is not necessary. For example, the microneedles can melt before and/or during their separation from the base, or the microneedles may not separate from the base at all.

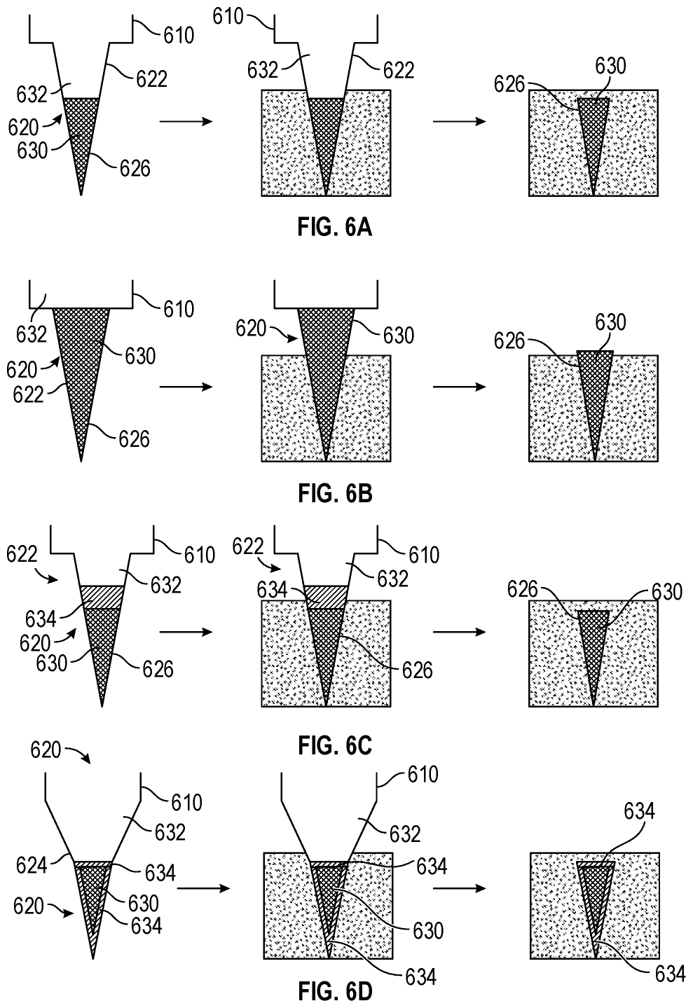

FIGS. 6A-6D illustrate some embodiments of methods of use. In FIG. 6A, the distal tip portion 626 of the microneedle 620 is formed of a drug-hydrophobic matrix composition 630 while the proximal end portion 622 of the microneedle 620 and the substrate 610 from which the microneedle extends are formed of a water-soluble composition 632, or an aqueous-cast base. When the microneedle 620 is inserted into the skin of a patient, the entire distal tip portion 626 is preferably inserted into the skin. While the distal tip 626 formed of the drug-hydrophobic matrix composition 630 is within the skin, this distal tip portion 626, and only this portion of the microneedle will melt upon being warmed by the patient's body temperature. The proximal end portion 622 also is partially inserted into the skin and can be solubilized in contact with interstitial fluid from the skin.

In FIG. 6B, the entire microneedle 622 is formed of a drug-hydrophobic matrix composition 630 and the base substrate 610 (from which the microneedle 620 extends) is formed of a water-soluble composition 632. When the microneedle 420 is inserted into the skin, at least the distal tip portion 626 of the microneedle 622 formed of the composition enters into the epidermal and possibly the dermal layers of the skin. However, at least some of the proximal end 622 of the microneedle 620 may remain outside of the skin. While inserted in the skin, at least a portion of the microneedle 620 will melt upon being warmed by the patient's skin, such that at least some of the drug-hydrophobic matrix composition 430 may release into the skin.

In FIG. 6C, the distal tip portion of the microneedle is formed of a drug-hydrophobic matrix composition 630, followed by a layer of an excipient composition 634, such as a fatty suppository base, then a base 632 forming the proximal end portion 622 of the microneedle 620 and the substrate 610 from which the microneedle 620 extends. While inserted, the excipient 634 layer will melt upon being heated by the patient's skin temperature, releasing the distal tip portion 626 into the skin. The drug-hydrophobic matrix composition 630 may or may not melt, or it may or may not dissolve, as part of releasing the drug into the skin.

In FIG. 6D, the distal tip portion 626 of the microneedle 620 is formed of a drug-hydrophobic matrix composition 630 and two excipient 634 casts, where the excipient 434 casts form a shell around the drug-hydrophobic matrix composition cast 630, as described with respect to FIG. 4I. The microneedle 620 also has a proximal base portion 624 formed of a water-soluble base 632, which also forms the substrate 610 from which the microneedle 620 extends. Similar to the embodiment of FIG. 6C, encasing the drug-hydrophobic matrix composition 630 in a shell of excipient 634 may be effective to delay release of the SOI, or limit the amount of SOI that is administered to the patient. The excipient 434 may also be effective as a hydrophobic barrier to protect the SOI composition 430 from being in contact with moisture that could affect the stability of the SOI composition 430.

In some embodiments, microneedles that do not melt into the skin may be formed of meltable excipients and or drug matrices. Use of meltable excipients to form microneedles may be beneficial when the active is not soluble, unstable, or risks damage at high temperatures. Forming microneedles out of meltable excipients reduces the temperature to which the drug-excipient matrix must be heating (i.e., at most about 100° C.), which will not damage biomolecules like certain proteins, vaccines, or DNA molecules. In some embodiments, meltable excipients may be used to reduce the cost of manufacturing microneedles by reducing the drying time for the microneedle patches.

For microneedles that do not melt in the skin, the active may be released accordingly to several mechanisms, such as diffusion, dissolution, and/or degradation. In embodiments, the microneedles may be manufactured such that the active is released through an intact microneedle. In other embodiments, the microneedle may be formed of an excipient configured to dissolve within the skin, such that the active is released into the skin as the microneedle dissolves. In further embodiments, the microneedle may be formed of an excipient configured to degrade through, for example, hydrolysis, enzymatic activity, or other similar mechanisms. This allows for release of the drug at a rate consistent with the rate of degradation. According to a preferred embodiment, the microneedle will dissolve or degrade so that it eventually disappears within the patient's skin.

In other embodiments, the active remains in a solid state (e.g., in the core of the microneedle or within a drug-excipient matrix), such that only the excipient melts within the skin. For example, certain actives (e.g., proteins, vaccines, DNA, etc.) are not soluble and therefore will not dissolve within the melted excipient during the casting process. So when the microneedle patch is applied to the skin, the excipient melts around the active such that it may be delivered within the skin.

This invention can be further understood with reference to the following non-limiting examples.

### EXAMPLE 1. SCREENING OF FATTY BASES AS MATERIALS FOR FORMING MICRONEEDLES

Several fatty base materials commonly used in the formulation of suppositories for rectal administration of drug were tested to determine their suitability for manufacturing microneedles and microneedle arrays (and therefore as a microneedle patch). The bases tested were Witepsol H15 (Fagron), 1% polyvinyl pyrrolidone (PVP) 360 kDa in Witepsol H15, 2% PVP 360 kDA in Witepsol H15, 1% poly(methyl methacrylate) (PMMA) 35 kDa in Witepsol H15, 1% polymethyl vinyl ether-alt-maleic anhydride (PMVE/MA) 216 kDa in Witepsol H15, 3% PMVE/MA 216 kDa in Witepsol H15, and 6% polycaprolactone (PCL) in Witepsol H15. Selection criteria for an optimal fatty base were the integrity and hardness of the resulting microneedle upon insertion into skin. All fatty base materials were tested with an 18% PVP (360/55)/18% sucrose second cast. Specifically, 10 μL of each fatty base material were cast into a microneedle mold at 50° C., and the molds were centrifuged at a relative centrifugal force of 3234×g and a temperature of 40° C. for 1 hour.

Of the fatty base materials tested, Witepsol H15 demonstrated optimal characteristics for a first cast material (forming the distal tip portions of the microneedles). After further optimization, it was subsequently determined that Witepsol S55 (IOI Oleo GmbH) was more suitable as it has the highest range of OH value (from about 50-65) among tested waxes.

### EXAMPLE 2. SCREENING OF NON-DISSOLVING OR WATER-SOLUBLE BACKING AS SECOND CAST

Various aqueous polymer-based formulations were tested for suitability as a second cast material (to form a proximal portion of the microneedles and/or the base from which the microneedles extend), based at least in part on compatibility with a fatty base microneedle (or distal tip portion thereof). All aqueous polymer-based formulations were tested using Witespol H15 as the first cast to form the microneedle, as described in Example 1.

The aqueous polymer-based formulations tested for the second-cast formulation included Witespol H15, 13% PVA/13% sucrose, 13% PVP 55 k Da, 13% PVP 360 k Da, and 13% PVP (360/55)/13% sucrose, and 18% PVP (360/55)/18% sucrose, as well as 20% polystyrene in dioxane and polycaprolactone in a melted state. Specifically, 400 μL of each polymer-based formulation was cast into a microneedle mold at 25° C., except Witespol H15 which was cast at 50° C. and polycaprolactone which was cast at 60° C. After being filled, the molds were placed on a vacuum chuck for 2 hours at 54° C., followed by an additional 2 hours at 25° C. The molds thereafter were removed from the vacuum chuck and left to dry before demolding the microneedle patches.

Of all the materials tested, it was determined that 13% PVP (360/55)/13% sucrose, and 18% PVP (360/55)/18% sucrose, but preferably 18% PVP (360/55)/18% sucrose, demonstrated the optimal characteristics for a second cast material.

### EXAMPLE 3. MICRONEEDLE INSERTION INTO SKIN

Porcine skin was obtained from a slaughterhouse, and the subcutaneous fat layer was removed using a dermatome. Sections were cleaned thoroughly with alcohol swabs, blotted dry with Kimwipes, affixed to a wooden block to support insertion, and placed in an oven set at 32° C. for 30 minutes to acquire the surrounding temperature. The wooden block was then removed from the oven briefly, and the microneedle patches were inserted into the skin. The skin, with the applied patches, was then placed back into the 32° C. oven for a maximum of 15 minutes. The microneedle patch was then removed, and the skin was microscopically imaged for signs of melting and microneedle separation from the base. The skin was also imaged using brightfield and fluorescence optics for the same purpose.

### EXAMPLE 4. LEVONORGESTREL-LOADED MICRONEEDLE PATCHES

Levonorgestrel-loaded melting microneedle patches were prepared with 60% drug loading and were characterized for in vitro release.

The levonorgestrel microneedle patch was placed in a nylon mesh bag (Nylon Monofilaments Mesh, Midwest Filter, St. Charles, IL) and then incubated in 1950 mL of 0.02% Tween 20 in phosphate-buffered saline (PBS) containing 25% ethanol as the release medium. The release experiment was carried out at 37° C. and with stirring at 80 rpm for 7 days. The 0.02% Tween 20 in PBS medium was first adjusted to pH 7.4, and then mixed with 25% ethanol for release medium. The nylon mesh bag with 1 μm pore size was used to better separate the released free drug from the molten microneedle matrices and undissolved levonorgestrel crystals. At predetermined time intervals, a 0.5 mL aliquot was collected from the release medium and replaced with the same volume of fresh media.

The amount of levonorgestrel released from each microneedle patch over time was quantitatively analyzed by ultra-performance liquid chromatography (UPLC, Waters, Milford, MA) equipped with a tunable ultra-violet (UV) detector. The levonorgestrel was separated on an Acquity UPLC Ethylene-Bridged Hybrid (BEH) C18 column (100 mm×2.1 mm inner diameter, 1.7 micrometer particle size) at 50° C. The mobile phase was a mixture of acetonitrile containing 0.1% formic acid and water containing 0.1% formic acid at a ratio of 55/45 (v/v) at a flow rate of 0.3 mL/min. The injection volume was 10 microliters. The UV absorbance of LNG was measured at 345 nm. The cumulative levonorgestrel release amounts (%) were calculated based on the levonorgestrel loading amount in the microneedles and then normalized based on the average amount released at the end of the experiment.

The cumulative release of levonorgestrel from the melting microneedle patch demonstrated a biphasic release pattern, having an initial zero-order burst followed by a roughly zero-order extended release for up to 7 days in 0.02% Tween 20 in PBS release media containing 25% (v/v) ethanol. The levonogrestrel melting microneedle patch achieved cumulative release of 100% within this 7 day time frame.

### EXAMPLE 5. MEASLES AND RUBELLA VACCINE LOADED MICRONEEDLE PATCHES

A higher melting point wax, specifically Witepsol E85, was used as a second middle cast between a first aqueous cast of a concentrated measles and rubella vaccine and a third aqueous cast of a PVA/sucrose composition, to form microneedle patches. The microneedle patches were then characterized for vaccine activity upon drying and stability upon storage at 40° C., using a TCID₅₀ assay.

Monolayers of Vero cells (ATCC, CCL-81) for measles and RK13 cells (ATCC, CCL-37) for rubella were seeded in 96-well plates at a density of 2.5×10⁴ cells/well and then inoculated with serial dilutions of reconstituted microneedle patches in Dulbecco's Modified Eagle Medium (DMEM). After 5 days of incubation at 37° C. and 5% CO₂, measles plates were stained with crystal violet solution for evidence of viral cytotoxic effect. For rubella plates, cells were fixed with methanol, and immunostaining was used to visualize the infected foci. Titers expressed as 50% tissue culture infectious dose (TCID₅₀) were calculated using the Spearman and Karber algorithm.

Some embodiments of the present disclosure can be described in view of one or more of the following:

Embodiment 1. A microneedle patch comprising a backing layer and an array of microneedles extending from the backing layer, the microneedles each comprising a distal tip portion which comprises a drug (or SOI), wherein the microneedles are configured to be inserted into mammalian tissue and at least a portion of each of the microneedles is configured to melt in the mammalian tissue.

Embodiment 2. The microneedle patch of Embodiment 1, wherein the mammalian tissue is human skin.

Embodiment 3. The microneedle patch of either of Embodiments 1 or 2, wherein the microneedle comprises the drug dissolved or dispersed in a wax, such as glycerol esters of fatty acids such as Witespol, or a fatty acid such as stearic acid, lauric acid, etc.

Embodiment 4. The microneedle patch of any one of Embodiments 1 to 3, wherein the microneedles are configured to separate from the backing layer following insertion into the mammalian tissue.

Embodiment 5. The microneedle patch of any one of Embodiments 1 to 4, wherein the drug is hydrophobic or hydrophilic.

Embodiment 6. The microneedle patch of any one of Embodiments 1 to 5, wherein the drug comprises a vaccine.

Embodiment 7. The microneedle patch of any one of Embodiments 1 to 6, wherein the drug comprises a protein, peptide, RNA or DNA.

Embodiment 8. The microneedle patch of any one of Embodiments 1 to 5, wherein the drug comprises a steroid or a hormone, such as levonorgestrel.

Embodiment 9. The microneedle patch of any one of Embodiments 1 to 8, wherein the microneedles further comprise a drug-free proximal portion between the backing layer and each of the distal tip portions.

Embodiment 10. The microneedle patch of Embodiment 9, wherein the drug-free proximal portion and/or the backing layer comprises one or more water soluble materials, for example polyivnylpyrrolidone, polyvinyl alcohol, a disaccharide, such as sucrose, or a combination thereof.

Embodiment 11. The microneedle patch of Embodiment 10, wherein the drug-free proximal portion comprises a wax.

Embodiment 12. A method of making a microneedle, the method comprising preparing a first composition comprising a drug (or SOI) dispersed in a first excipient material, such as wax, heated to a temperature above its melting point, casting the first composition at a temperature less than 100° C. (e.g., less than 90, 80, 70, 60, 50, or 40° C.) in a mold having a cavity defining one or more microneedles, and cooling the first composition in the mold to below the melting point of the first excipient material to form one or more microneedles, or at least distal tip portions thereof, in the microneedle mold.

Embodiment 13. The method of Embodiment 12, further comprising casting a second composition onto the distal tip portion of the one or more microneedles to form a proximal portion of the one or more microneedles.

Embodiment 14. The method of Embodiment 13, wherein the second composition comprises a solution comprising an aqueous solvent and solute materials, for example, polyvinylpyrrolidone, polyvinyl alcohol, a disaccharide, such as sucrose, or a combination thereof.

Embodiment 15. The method of Embodiment 13, wherein the second composition is solvent-free and comprises a second excipient material, such as wax, heated to a temperature above its melting point.

Embodiment 16. The method of any one of Embodiments 12 to 15, wherein the first excipient and/or the second excipient comprises a glycerol ester of fatty acids, such as Witepsol, or a fatty acid such as stearic acid, lauric acid, etc.

Embodiment 17. The method of any one of Embodiments 12 to 16, further comprising casting a backing layer onto a base of the one or more microneedles.

Embodiment 18. The method of any one of Embodiments 12 to 17, wherein the drug is hydrophobic or wherein the drug is hydrophilic.

Embodiment 19. The method of any one of Embodiments 12 to 17, wherein the drug comprises a vaccine.

Embodiment 20. The method of any one of Embodiments 12 to 17, wherein the drug comprises a protein, a peptide, RNA or DNA.

Embodiment 21. The method of any one of Embodiments 12 to 17, wherein the drug comprises a steroid or a hormone, such as levonorgestrel.

Embodiment 22. The microneedle patch of any one of Embodiments 1 to 11, or the method of any one of Embodiments 12 to 21, wherein the microneedle is made of one or more materials that melt at a temperature from 20° C. to 50° C. or from 30° C. to 37° C., such as 32° C. to 35° C.

Embodiment 23. The microneedle patch of any one of Embodiments 1 to 11, or the method of any one of Embodiments 12 to 21, wherein the microneedle is made of one or more materials that melt at a temperature from 38° C. to 100° C., such as 40° C. to 90° C., 40° C. to 60° C., or 40° C. to 50° C.

Embodiment 24. The microneedle patch of any one of Embodiments 1 to 11, 22, and 23, wherein the portion of each of the microneedles configured to melt in the mammalian tissue comprises, or consists of, the distal tip portion.

Embodiment 25. The microneedle patch of any one of Embodiments 1 to 11, 22, and 23, wherein the portion of each of the microneedles configured to melt in the mammalian tissue comprises, or consists of, a (or the) drug-free proximal portion.

Embodiment 26. The microneedle patch of any one of Embodiments 1 to 11 and 22 to 25, wherein the backing layer is configured to melt in contact with the mammalian tissue.

Embodiment 27. The microneedle patch or any one of Embodiments 1 to 11 and 22 to 26, wherein a (or the) drug-free proximal portion of each of the microneedles is formed of one or more hydrophobic materials having a melting point from 38° C. to 60° C., such as 40° C. to 50° C.

Embodiment 28. The microneedle patch of Embodiment 27, wherein the distal tip portion of each of the microneedles and/or the backing layer is formed of one or more hydrophilic materials.

Embodiment 29. The method of any one of Embodiments 12 to 21, wherein the drug is in a solid state and is non-homogenously mixed with the first excipient material.

Embodiment 30. A method of delivering a drug to a patient, the method comprising inserting one or more microneedles, which comprise a drug, into the patient's skin, and then melting the one or more microneedles, or at least a portion thereof, (i) to release the drug into the patient's skin, and/or (ii) to separate at least a portion of the one or more microneedles from a base, or backing layer, of a microneedle patch.

Embodiment 31A. The method of Embodiment 30, wherein the melting occurs when the inserted one or more microneedles reach the body temperature of the patient (e.g., 30° C. to 37° C.).

Embodiment 31B. The method of Embodiment 30, wherein the melting comprises application of heat from a source external to the patient to the inserted one or more microneedles, wherein the applied heat is delivered in an amount/rate effective to cause at least a portion of the microneedles to melt.

Embodiment 32. The method of any one of Embodiments 29 to 31, wherein the one or more microneedles are part of a microneedle patch comprising an array of microneedles extending from a backing layer.

Embodiment 33. The method of Embodiment 32, further comprising separating the microneedles from the backing layer after the inserting.

Embodiment 34. The method of any one of Embodiments 29 to 33, wherein the melted microneedles in the skin form a hydrophobic liquid depot from which the drug is released over an extended period of time.

Embodiment 35. The method of Embodiment 34, wherein the extended period is at least one day, for example, 2 days, 3 days, 4 days, one week, two weeks, three weeks, four weeks, or one month.

Embodiment 36. A method of segregating a drug in a distal tip portion of a microneedle in a microneedle patch from a backing layer of the microneedle patch by interposing a hydrophobic material, such as a wax, in proximal portion of the microneedles between the backing layer and the distal tip portion.

Embodiment 37. The method of Embodiment 36, wherein the drug is a vaccine or other biological molecule and/or is sensitive to degradation by exposure to moisture.

Embodiment 38. The method of either of Embodiments 36 or 37, wherein the distal tip portion is configured to dissolve or melt following insertion into tissue (e.g., mammalian skin or other biological tissue).

Embodiment 39. The method of any one of Embodiments 36 to 38, wherein the proximal portion is configured to melt following insertion into tissue (e.g., mammalian skin or other biological tissue).

Embodiment 40. The method of any one of Embodiments 29 to 39, wherein the proximal portion is formed of one or more materials, such as a wax, that have a melting point from 38° C. to 100° C., e.g., between 50° C. and 100° C.

Embodiment 41. The method of any one of Embodiments 29 to 40, wherein the distal tip portion is formed of one or more materials that have a melting point from 20° C. to 50° C.

Embodiment 42. A microneedle patch comprising: a backing layer; and an array of microneedles extending from the backing layer, the microneedles each comprising a portion which comprises a drug; wherein the microneedles are configured to be inserted into mammalian tissue, and wherein the microneedles are made of one or more materials that melt at a temperature between 60° C. and 100° C. (e.g., between 70° C. and 100° C.).

Embodiment 43. The microneedle patch of Embodiment 42, wherein the microneedles are configured to dissolve or bioerode in the mammalian skin.

Embodiment 44. A method of delivering a drug to a patient, the method comprising: inserting a microneedle, which comprises a drug, into the patient's skin; and then melting the microneedle, or a distal tip portion thereof, into the patient's skin.

Embodiment 45. The method of Embodiment 44, wherein the melting occurs when the inserted microneedle reaches the skin temperature of the patient.

Embodiment 46. The method of Embodiment 44, wherein the melting comprises application of heat from a source external to the patient to the inserted microneedle.

Embodiment 47. The method of any one of Embodiments 44 to 46, wherein the microneedle is part of a microneedle patch comprising an array of microneedles extending from a backing layer.

Embodiment 48. The method of any one of Embodiments 44 to 47, further comprising: separating the microneedle from the backing layer after the inserting.

Embodiment 49. The method of any one of Embodiments 44 to 48, wherein the melted microneedles in the skin form a hydrophobic liquid depot from which the drug is released over an extended period of at least one day.

Embodiment 50. The method of Embodiment 49, wherein the extended period is from 2 days to 30 days.

The term “about,” as used herein, indicates the value of a given quantity and can include quantities ranging within 10% of the stated value, or optionally, within 5% of the value, or in some embodiments, within 1% of the value.

While the disclosure has been described with reference to a number of exemplary embodiments, it would be understood by those skilled in the art that the disclosure is not limited to such disclosed embodiments. Rather, the disclosed embodiments can be modified to incorporate any number of variations, alterations, substitutions, or equivalent arrangements not described herein, but which are commensurate with the spirit and scope of the disclosure.

1. A microneedle patch comprising:
a backing layer; and
an array of microneedles extending from the backing layer, the microneedles each comprising a distal tip portion which comprises a drug;
wherein the microneedles are configured to be inserted into mammalian tissue and at least a portion of each of the microneedles is configured to melt in the mammalian tissue.

2. The microneedle patch of claim 1, wherein the mammalian tissue is human skin.

3. The microneedle patch of claim 1, wherein the microneedle comprises the drug dissolved or dispersed in a wax.

4. The microneedle patch of claim 3, wherein the wax comprises a fatty acid or a glycerol ester of a fatty acid.

5. The microneedle patch of claim 3, wherein the wax comprises stearic acid, lauric acid, or another fatty acid.

6. The microneedle patch of claim 1, wherein the microneedles, or at least a portion of the distal tip portions thereof, are configured to separate from the backing layer following insertion into the mammalian tissue.

7. The microneedle patch of claim 1, wherein the portion of each of the microneedles that is configured to melt in the mammalian tissue comprises at least a portion of the distal tip portion.

8. The microneedle patch of claim 1, wherein the portion of each of the microneedles that is configured to melt in the mammalian tissue comprises a drug-free proximal portion.

9. The microneedle patch of claim 1, wherein the backing layer is configured to melt in contact with the mammalian tissue.

10. The microneedle patch of claim 1, wherein the drug is hydrophobic.

11. The microneedle patch of claim 1, wherein the drug comprises a vaccine.

12. The microneedle patch of claim 1, wherein the drug comprises levonorgestrel or another steroid or hormone.

13. The microneedle patch of claim 1, wherein the drug comprises a protein, peptide, RNA or DNA.

14. The microneedle patch of claim 1, wherein the microneedles further comprise a drug-free proximal portion between the backing layer and each of the distal tip portions.

15. The microneedle patch of claim 14, wherein the drug-free proximal portion and/or the backing layer comprises one or more water-soluble materials.

16. The microneedle patch of claim 14, wherein the drug-free proximal portion comprises a wax.

17. The microneedle patch of claim 14, wherein the drug-free proximal portion of each of the microneedles is formed of one or more hydrophobic materials having a melting point from 20° C. to 50° C.

18. The microneedle patch of claim 17, wherein the distal tip portion of each of the microneedles and/or the backing layer is formed of one or more hydrophilic materials.

19. A method of making a microneedle, the method comprising:
preparing a first composition comprising a drug dispersed in a first excipient material heated to a temperature greater than the melting point of the first excipient material;
casting the first composition at a temperature less than 100° C. in a mold having a cavity defining one or more microneedles; and
cooling the first composition in the mold to a temperature that is less than the melting point of the first excipient material to form one or more microneedles, or at least a distal tip portion of one or more microneedles, in the mold.

20. The method of claim 19, further comprising:
casting a second composition onto the distal tip portion of the one or more microneedles to form a proximal portion of the one or more microneedles.

21. The method of claim 20, wherein the second composition comprises a solution comprising an aqueous solvent and solute materials.

22. The method of claim 20, wherein the second composition is solvent-free and comprises a second excipient material heated to a temperature greater than the melting point of the second excipient material.

23. The method of claim 22, wherein the first excipient and/or the second excipient comprises a fatty acid or a glycerol ester of a fatty acid.

24. The method of claim 19 further comprising:
casting a backing layer onto a base of the one or more microneedles.

25-28. (canceled)

29. The method of claim 19, wherein the drug is in a solid state and is non-homogenously mixed with the first excipient material.

30. The microneedle patch of claim 1, wherein the microneedle is made of one or more materials that melt at a temperature from 20° C. to 50° C.

31-32. (canceled)

33. A microneedle patch comprising:
a backing layer; and
an array of microneedles extending from the backing layer, the microneedles each comprising a portion which comprises a drug;
wherein the microneedles are configured to be inserted into mammalian tissue, and
wherein the microneedles are made of one or more materials that melt at a temperature between 60° C. and 100° C.

34. The microneedle patch of claim 33, wherein the microneedles are configured to dissolve or bioerode in the mammalian skin.

35. A method of delivering a drug to a patient, the method comprising:
inserting a microneedle, which comprises a drug, into the patient's skin; and then melting the microneedle, or a distal tip portion thereof, into the patient's skin.

36-39. (canceled)

40. The method of claim 35, wherein the melted microneedles in the skin form a hydrophobic liquid depot from which the drug is released over an extended period of at least one day.

41. The method of claim 40, wherein the extended period is from 2 days to 30 days.

42. A method of segregating a drug in a distal tip portion of a microneedle in a microneedle patch from a backing layer of the microneedle patch, the method comprising interposing a hydrophobic material in a proximal portion of the microneedle between the backing layer and the distal tip portion.

43-47. (canceled)
